(19) United States
(12) Patent Application Publication
(10) Pub. No.: US 2005/0031574 A1
(43) Pub. Date: Feb. 10, 2005

(54) Cosmetic composition comprising polymers having a star structure, the polymers, and their use

(75) Inventor: Nathalie Mougin, Paris (FR)

Correspondence Address: FINNEGAN, HENDERSON, FARABOW, GARRETT & DUNNER;LLP, 1300 I STREET, NW, WASHINGTON, DC, 20005, US

(73) Assignee: L'OREAL

(21) Appl. No.: 10/656,238

(22) Filed: Sep. 8, 2003

Related U.S. Application Data
(62) Division of application No. 09/544,655, filed on Apr. 5, 2000, now abandoned.

(30) Foreign Application Priority Data
Apr. 6, 1999 (FR) 99 04258

(51) Int. Cl. A61K 31/74; C08L 1/00

(52) U.S. Cl. 424/78.1; 525/185

(57) ABSTRACT

A composition capable of being applied to the hair, comprising, in a cosmetically acceptable medium, at least one polymer with a highly specific ordered structure is disclosed. A process for shaping or retaining the form of the hair using this composition and the use in the formulation of styling products, such as lacquers, sprays or foams, for the purpose of obtaining form retention or shaping of the hairstyle is also disclosed.

The present invention relates to a composition capable of being applied to the hair, in particular a styling composition, comprising, in a cosmetically acceptable medium, at least one polymer with a highly specific ordered structure. It is also targeted at a process for shaping or retaining the form of the hair using this composition and at its use in the formulation of styling products, such as lacquers, sprays or foams, for the purpose of obtaining form retention or shaping of the hairstyle.

The most widespread hair products on the cosmetics market for fixing the hair are compositions to be sprayed as an aerosol or as a pump-action spray, such as lacquers, sprays or foams, composed mainly of a solution, generally an alcoholic or aqueous/alcoholic solution, and of a water-soluble or alcohol-soluble film-forming polymer, as a mixture with various cosmetic adjuvants.

However, these hair formulations, such as foams, gels and especially aerosol sprays and lacquers intended to retain the form of the hairstyle, do not make it possible for the hairstyle to satisfactorily withstand the various natural movements of life, such as walking, head movements or gusts of wind. This is because the polymers used for the formulation of these hair products are generally anionic, amphoteric or non-ionic film-forming polymers which result in the formation of films having a more or less hard and brittle nature. When the polymer is too brittle, the percentage of elongation at break measured on the film is low, that is to say generally of less than 2%, and the hold of the hairstyle over time is not assured.

To overcome this problem, these polymers have already been mixed with plasticizers, and more flexible and non-flaky coatings have already been obtained. However, these films are deformable and plastic, that is to say that, after deformation, they only recover their initial form to a very small extent. While the hold of the hairstyle is improved, it is still not satisfactory since the form of the hairstyle changes over time.

More satisfactory results in terms of hold have been obtained with compositions comprising a combination of film-forming polymers, such as, for example, a cellulose polymer and an acrylic polymer. However, these compositions are still not entirely satisfactory, insofar as the hair loses some of its natural cosmetic properties.

There is therefore a search for cosmetic compositions for form retention of and/or for fixing the hairstyle which provide the hair with good cosmetic properties, in particular good disentangling, softness and a pleasant appearance, in addition to fixing which lasts.

The Inventor has found that, surprisingly and unexpectedly, the use of highly specific polymers exhibiting a specific ordered structure can make it possible to obtain a composition capable of being applied to keratinous fibres, in particular the hair, which can make possible suitable form retention of the hair while retaining ease of disentangling and a pleasant appearance of the hair.

Thus, a subject-matter of the present invention is a polymer with a “star” structure represented by the following formula (I):
A−[(M1)ₚ₁−(M2)ₚ₂ . . . (Mi)ₚⱼ]ₙ
in which:
A represents a polyfunctional centre, with a functionality of “n”, n being an integer greater than or equal to 2, preferably of between 4 and 10,
[(M1)ₚ₁−(M2)ₚ₂ . . . (Mi)ₚⱼ] represents a polymer chain, also known as a “branch”, composed of identical or different polymerized monomers Mi having a polymerization index pj, each branch being identical or different and being grafted covalently to the centre A;
i being greater than or equal to 2 and pj being greater than or equal to 2;
the polymer comprising one or more polymerized monomers Mi chosen from polymerized monomers Mk, which may be identical or different, wherein a homopolymer formed by the corresponding polymerized monomers Mk exhibits a Tg of greater than or equal to approximately 10° C., preferably of greater than or equal to 15° C. and even better still of greater than or equal to 20° C.; this or these monomers Mi being present, in the final polymer, in an amount of between 55 and 95% by weight, preferably in an amount of 60 to 93% by weight and even better still in an amount of 65-90% by weight with respect to the total weight of monomers; and
the polymer furthermore comprising one or more polymerized monomers Mj chosen from polymerized monomers Mj, which may be identical or different, wherein a homopolymer formed by the corresponding polymerized monomers Mj exhibits a Tg of less than or equal to approximately 10° C., preferably of less than or equal to 5° C. and even better still of less than or equal to 0° C.; this or these monomers Mj being present, in the final polymer, in an amount of between 5 and 45% by weight, preferably in an amount of 7 to 40% by weight and even better still in an amount of 10-35% by weight with respect to the total weight of monomers.

Another subject-matter of the invention is a composition comprising, in a cosmetically acceptable medium, at least one polymer as defined above.

Another subject-matter of the invention is a process for form retention of or shaping the hair, characterized in that it comprises applying, to the latter, a cosmetic composition as defined above.

Another subject-matter of the invention is the use of a cosmetic composition as defined above in the manufacture of a hair cosmetic product intended to retain the form of and/or to fix and/or to treat the hairstyle and/or the hair.

The composition according to the invention therefore comprises a polymer, the “star” structure of which can be illustrated, in a general way, by the following formula (I):
A−[(M 1)ₚ₁−(M2)ₚ₂ . . . (Mi)ₚⱼ]ₙ
in which:
A represents a polyfunctional centre, with a functionality of “n”, n being an integer greater than or equal to 2, preferably of between 4 and 10,
[(M 1)ₚ₁−(M2)ₚ₂ . . . (Mi)ₚⱼ] represents a polymeric chain, also known as a “branch”, composed of identical or different polymerized monomers Mi having a polymerization index pj, each branch being identical or different and being grafted covalently to the centre A;
i being greater than or equal to 2, preferably of between 2 and 10; and
pj being greater than or equal to 2, preferably of between 10 and 20,000.

The polymer chains are preferably provided in the form of blocks with a molecular mass of greater than or equal to 500 which can range up to 2,000,000.

In a preferred embodiment, the polymer used in the context of the present invention can be obtained by controlled radical polymerization, also known as “living” radical polymerization. This technique makes it possible in particular to overcome the limitations inherent in conventional radical polymerization, that is to say that it makes it possible in particular to control the length of the chains of the polymer which is formed and therefore to obtain block structures.

The controlled radical polymerization makes it possible to reduce the reactions in which the growing radical species is deactivated, in particular the termination stage, which reactions, in conventional radical polymerization, interrupt the growth of the polymer chain in an irreversible and uncontrolled way.

In order to decrease the probability of termination reactions, provision has been made to block, in a temporary and reversible way, the growing radical species by forming so-called “dormant” active species with the aid of a bond of low 101 dissociation energy.

In particular, mention may be made of the possibility of using bonds of C—ONR type (by reaction with a nitroxyl); this is illustrated in particular by the article “Synthesis of nitroxy-functionalized polybutadiene by anionic polymerization using a nitroxy-functionalized terminator”, published in Macromolecules, 1997, volume 30, pp. 4238-4242.

Mention may also be made of the possibility of using bonds of C-halide type (in the presence of metal/ligand complex). This is then described as atom transfer radical polymerization, also known under the abbreviation ATRP. This type of polymerization is reflected in control of the mass of the polymers which are formed and in a low polydispersity index by weight of the chains.

Atom transfer radical polymerization is generally carried out by polymerization:
of one or more radically polymerizable monomers, in the presence
of an initiator having at least one radically transferable atom or group,
of a compound comprising a transition metal capable of participating in a reduction stage with the initiator and a “dormant” polymer chain, and
of a ligand, which can be chosen from compounds comprising a nitrogen (N), oxygen (O), phosphorus (P) or sulphur (S) atom, which compounds are capable of coordinating via a σ bond to the compound comprising a transition metal, or from compounds comprising a carbon atom, which compounds are capable of coordinating via a π or σ bond to the compound comprising a transition metal, the formation of direct bonds between the compound comprising a transition metal and the polymer in the course of formation being avoided.

This process is illustrated in particular in Application WO97/18247, the teaching of which can be drawn upon by a person skilled in the art in preparing the polymers coming within the scope of the present invention.

The nature and the amount of the monomers, initiator(s), compound(s) comprising the transition metal and ligand(s) will be chosen by a person skilled in the art on the basis of his overall knowledge, according to the result desired.

In particular, the monomers “M” (Mi, Mk, and Mj) can be chosen, alone or as a mixture, from radically polymerizable compounds comprising ethylenic unsaturation corresponding to the formula: 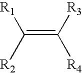
in which R₁, R₂, R₃ and R₄ are, independently of one another, chosen from:
a hydrogen atom;
a halogen atom;
a linear or branched alkyl radical having 1 to 20, preferably 1-6, more preferably 1-4, carbon atoms which is optionally substituted by one or more halogens and/or one or more —OH radicals;
a linear or branched alkenyl or alkynyl radical having 2 to 10, preferably 2-6, more preferably 2-4, carbon atoms which is optionally substituted by one or more halogens;
a cyclic hydrocarbonaceous (cycloalkyl) radical having 3 to 8 carbon atoms which is optionally substituted by one or more halogen, nitrogen, sulphur or oxygen atoms;
a radical chosen from CN, C(═Y)R⁵, C(═Y)NR⁶R⁷, YC(═Y)R⁵, cyclic NC(═Y)R⁵ SOR⁵, SO₂R⁵ OSO₂R⁵, NR⁸SO₂R⁵, PR⁵₂, P(═Y)R⁵₂, YPR⁵₂, YP(═Y)R⁵₂, NR⁸₂, which can be quaternized with an additional R⁸ group, aryl and heterocyclyl, with:
   Y represents O, S or NR⁸ (preferably O),
   R⁵ represents a linear or branched alkyl, alkylthio or alkoxy radical having 1-20 carbon atoms; an OH radical; an OM′ radical with M′=alkali metal; an aryloxy radical or a heterocyclyloxy radical;
   R¹ and R⁷ represent, independently of one another, H or a linear or branched alkyl radical having 1-20 carbon atoms; it being given that R⁶ and R⁷ can be joined to form an alkylene group having 2-7, preferably 2-5, carbon atoms;
   R⁸ represents H, a linear or branched alkyl radical having 1-20 carbon atoms or an aryl radical;
a —COOR radical, in which R is a linear or branched alkyl radical having 1 to 20, preferably 1-6, carbon atoms which is optionally substituted by one or more halogens;
a —CONHR′ radical, in which R′ is hydrogen or a saturated or unsaturated, linear or branched, hydrocarbonaceous radical having 1 to 20, preferably 1-6, carbon atoms which is optionally substituted by one or more halogens, nitrogens and/or oxygens;
an —OCOR″ radical, in which R″ is hydrogen or a saturated or unsaturated, linear or branched, hydrocarbonaceous radical having 1 to 20 carbon atoms which is optionally substituted by one or more halogens, nitrogens and/or oxygens;
a radical comprising at least one silicon atom and in particular radicals such as: an —R-siloxane radical, a —CONHR-siloxane radical, a —COOR-siloxane radical or an —OCO—R-siloxane radical, in which radicals R is a linear or branched alkyl, alkylthio, alkoxy, aryloxy or heterocycloxy radical having 1-20 carbon atoms.

The term “siloxane” is understood to mean a compound comprising (—SiR^{a}R^{b}O—)ₙ units, in which units R^{a} and R^{b} can represent, independently of one another, a hydrogen; a halogen; a saturated or unsaturated, linear or branched, hydrocarbonaceous radical having 1 to 36 carbon atoms which is optionally substituted by one or more halogens, nitrogens and/or oxygens; or a cyclic hydrocarbonaceous radical having 1 to 20 carbon atoms; n being greater than or equal to 1.

For the purpose of this invention, the term “independent,” when used to describe the relationship of radicals, atoms, substituents, functional groups, etc., means that each of the radicals, atoms, substituents, functional groups, etc. may be the same or different from the other, or some radicals, atoms, substituents, functional groups, etc., may be the same while the others may be different.

Mention may in particular be made of polydimethylsiloxanes (PDMSs) comprising 1 to 200, preferably less than 100, repeat units.

Furthermore, R¹ and R³ can be connected to one another so as to form a ring of formula (CH₂)ₙ which can be substituted by one or more halogens and/or oxygens and/or nitrogens and/or by alkyl radicals having 1 to 6 carbon atoms.

The term “aryl” or “heterocyclyl” is understood to mean the definition commonly understood by a person skilled in the art and which may be illustrated by the prior art WO97/18247.

Preferably, the monomers M can be chosen from:
acrylic or methacrylic esters obtained from linear, branched or cyclic aliphatic alcohols and/or from aromatic alcohols, preferably C₁-C₂₀ alcohols, such as methyl (meth)acrylate, ethyl (meth)acrylate, propyl (meth)acrylate, butyl (meth)acrylate, isobutyl (meth)acrylate or tert-butyl (meth)acrylate;
C₁-C₄ hydroxyalkyl (meth)acrylates, such as 2-hydroxyethyl (meth)acrylate or 2-hydroxypropyl (meth)acrylate;
ethylene glycol, diethylene glycol or polyethylene glycol (meth)acrylates with a hydroxyl or ether end;
vinyl, allyl or methallyl esters obtained from linear or branched C₁-C₁₀ or cyclic C₁-C₆ aliphatic alcohols and/or from aromatic alcohols, preferably C₁-C₆ alcohols, such as vinyl acetate, vinyl propionate, vinyl benzoate or vinyl tert-butylbenzoate;
N-vinylpyrrolidone; vinylcaprolactam; vinyl-N-alkylpyrroles having 1 to 6 carbon atoms; vinyloxazoles; vinylthiazoles; vinylpyrimidines; vinylimidazoles; and vinyl ketones;
(meth)acrylamides obtained from linear, branched or cyclic aliphatic amines and/or from aromatic amines, preferably C₁-C₂₀ amines, such as tert-butylacrylamide; and (meth)acrylamides, such as acrylamide, methacrylamide or di(C₁-C₄)alkyl(meth)acrylamides;
olefins, such as ethylene, propylene, styrene or substituted styrene;
fluorinated or perfluorinated acrylic or vinyl monomers, in particular (meth)acrylic esters with perfluoroalkyl units;
monomers comprising an amine functional group in the free or else partially or completely neutralized or else partially or completely quaternized form, such as dimethylaminoethyl (meth)acrylate, dimethylaminoethylmethacrylamide, vinylamine, vinylpyridine or diallyldimethylammonium chloride;
carboxybetaines or sulphobetaines obtained by partial or complete quaternization of monomers comprising ethylenic unsaturation comprising an amine functional group by sodium salts of carboxylic acids comprising a mobile halide (sodium chloroacetate, for example) or by cyclic sulphones (propane sulphone);
silicone-comprising (meth)acrylates or (meth)acrylamides, in particular (meth)acrylic esters comprising siloxane units;
-their mixtures.

The particularly preferred monomers are chosen from:
(meth)acrylic esters obtained from linear or branched aliphatic alcohols, preferably C₁-C₂₀ alcohols;
C₁-C₂₀ (meth)acrylic esters comprising perfluoroalkyl units;
C₁-C₂₀ (meth)acrylic esters comprising siloxane units;
(meth)acrylamides obtained from linear, branched or cyclic aliphatic amines and/or from aromatic amines, preferably C₁-C₂₀ amines, such as tert-butylacrylamide; or (meth)acrylamides, such as acrylamide, methacrylamide or di(C₁-C₄)alkyl(meth)acrylamides;
vinyl, allyl or methallyl esters obtained from linear or branched C₁-C₁₀ or cyclic C₁-C₆ aliphatic alcohols;
vinylcaprolactam;
optionally substituted styrene;
their mixtures.

In the context of the present invention, the initiator can be any compound, in particular a molecular or polymeric compound, having at least two atoms and/or groups which are radically transferable by polymerization.

The initiator can in particular be an oligomer or a polymer capable of being obtained by radical polymerization, by polycondensation, by anionic or cationic polymerization or by ring opening.

The transferable atoms and/or groups can be situated at the ends of the polymer chain or along the backbone.

Mention may in particular be made of the compounds corresponding to one of the following formulae:
R¹¹CO—X
R¹¹ₓR¹²_{y}R¹³_{z}C—(RX)ₜ, in which x, y and z represent an integer ranging from 0 to 4, t an integer ranging from 1 to 4, and x+y+z=4-t;
R¹³ₓC₆—(RX)_{y} (saturated ring with 6 carbons), in which x represents an integer ranging from 7 to 11, y represents an integer ranging from 1 to 5, and x+y=12;
R¹³ₓC₆—(RX)_{y} (unsaturated ring with 6 carbons), in which x represents an integer ranging from 0 to 5, y represents an integer ranging from 1 to 6, and x+y=6;
[—(R¹¹)(R¹²)(R¹³)C—(RX)—]ₙ, in which n is greater than or equal to 1; cyclic or linear;
[—(R¹²)ₓC₆(RX)_{y}—R¹¹—]ₙ, in which x represents an integer ranging from 0 to 6, y represents an integer ranging from 1 to 6 and n is greater than or equal to 1, with x+y=4 or 6; cyclic or linear;
[—(R¹²)ₓC₆(RX)_{y}—R¹¹—]ₙ, in which x represents an integer ranging from 0 to 12, y represents an integer ranging from 1 to 12 and n is greater than or equal to 1, with x+y=10 or 12; cyclic or linear;
R¹¹R¹²R¹³Si—X
—[OSi(R¹¹)ₓ(RX)_{y}]ₙ, cyclic or linear, in which x and y represent an integer ranging from 0 to 2 and n is greater than or equal to 1, with x+y 2;
R¹¹R¹²N—X
R¹¹N—X₂
(R¹¹)ₓP(O)_{y}—X₃₋ₓ, in which x and y represent integers ranging from 0 to 2 and x+y=5;
(R¹¹O)ₓP(O)_{y}—X₃₋ₓ, in which x and y represent integers ranging from 0 to 2 and x+y=5;
—[(R¹¹)ₜN_{z}P(O)ₓ(O—RX)_{y}—]ₙ, cyclic or linear, in which x represents an integer ranging from 0 to 4, y represents an integer ranging from 1 to 5, z represents an integer ranging from 0 to 2, t represents an integer ranging from 0 to 3 and n is greater than or equal to 1;
   in which:
R, R¹¹, R¹² and R¹³ represent, independently of one another, a hydrogen or halogen atom; a linear or branched alkyl radical having 1-20, preferably 1-10 and more preferably 1-6 carbon atoms; a cycloalkyl radical having 3-8 carbon atoms; a —C(═Y)R⁵, —C(═Y)NR⁶R⁷ or —R⁸₃Si radical (see the definitions of R⁵ to R⁸ above);
COCI; —OH; —CN; an alkenyl or alkynyl radical having 2-20, preferably 2-6, carbon atoms; an oxiranyl or glycidyl radical or an alkylene or alkenylene radical substituted with an oxiranyl or a glycidyl; an aryl, heterocyclyl, aralkyl or aralkenyl radical; or an alkyl radical having 1-6 carbon atoms in which all or part of the hydrogen atoms are substituted either by halogen atoms, such as fluorine, chlorine or bromine, or by an alkoxy group having 14 carbon atoms or by an aryl, heterocyclyl, —C(═Y)R⁵, —C(═Y)NR⁶R⁷, oxiranyl or glycidyl radical;
X represents a halogen atom, such as Cl, Br or I, or an —OR′, —SR, —SeR, —OC(═O)R′, —OP(═O)R′, —OP(═O)(OR′)₂, —OP(═O)OR′, —O—NR′₁₂, —S—C(═S)NR′₂, —CN, —NC, —SCN, —CNS, —OCN, —CNO and —N₃ radical, in which R′ represents an alkyl radical having 1-20 carbon atoms which is optionally substituted by one or more halogen atoms, in particular fluorine and/or chlorine atoms, and R represents a linear or branched alkyl or aryl radical having 1-20, preferably 1-10, carbon atoms, it additionally being possible for the —NR′₂ group to represent a cyclic group, the two R′ groups being joined so as to form a 5-, 6- or 7-membered heterocycle.

Preferably, X represents a halogen atom and in particular a chlorine or bromine atom.

The initiator is preferably chosen from the compounds of formula —R¹³ₓC₆—(RX)_{y} (saturated ring with 6 carbons) in which x represents an integer ranging from 7 to 11, y represents an integer ranging from 1 to 5 and x+y=12;
—[—(R¹²)ₓC₆(RX)_{y}—R¹¹—]ₙ, in which x represent an integer ranging from 0 to 6, y represents an integer ranging from 1 to 6 and n is greater than or equal to 1, with x+y=4 or 6; cyclic or linear; and
—[OSi(R¹¹)ₓ(RX_{y}]ₙ, cyclic or linear, in which x and y represent an integer ranging from 0 to 2 and n is greater than or equal to 1, with x+y=2.

Mention may in particular be made, as initiator, of the following compounds:
octa(2-isobutyrylbromide)octa(tert-butyl)calix(8)arene,
octa(2-propionylbromide)octa(tert-butyl)calix(8)arene, and
hexakis(α-bromomethyl)benzene.

The compound comprising a transition metal which is capable of participating in a reduction stage with the initiator and a “dormant” polymer chain can be chosen from those which correspond to the formula Mⁿ⁺X′ₙ, in which formula:
M can be chosen from Cu, Au, Ag, Hg, Ni, Pd, Pt, Rh, Co, Ir, Fe, Ru, Os, Re, Mn, Cr, Mo, W, V, Nb, Ta and Zn,
X′ can represent a halogen (in particular bromine or chlorine), OH, (O)_{1/2}, an alkoxy radical having 1-6 carbon atoms, (SO₄)_{1/2}, (PO₄)_{1/3}, (HPO₄)_{1/2}, (H₂PO₄), a triflate, hexafluorophosphate, methanesulphonate, arylsulphonate, SeR, CN, NC, SCN, CNS, OCN, CNO, N₃ and R′CO₂ radical, in which R represents a linear or branched alkyl or aryl radical having 1-20, preferably 1-10, carbon atoms and R′ represents H or a linear or branched alkyl radical having 1-6 carbon atoms or an aryl radical which is optionally substituted by one or more halogen atoms, in particular fluorine and/or chlorine atoms;
n is the charge on the metal.

The choice is preferably made of M representing copper or ruthenium and X′ representing bromine or chlorine.

Mention may in particular be made of copper bromide.

Mention may be made, among the ligands capable of being used in the context of the present invention, of compounds comprising at least one nitrogen, oxygen, phosphorus and/or sulphur atom which are capable of coordinating via a a bond to the compound comprising a transition metal.

Mention may also be made of compounds comprising at least two carbon atoms which are capable of coordinating via a T bond to the compound comprising a transition metal.

Mention may further be made of compounds comprising at least one carbon atom which are capable of coordinating via a σ bond to the compound comprising a transition metal but which do not form a carbon-carbon bond with the monomer during the polymerization, that is to say which do not participate in β-addition reactions with the monomers.

Mention may further be made of compounds capable of coordinating via μ or η bond to the compound comprising a transition metal.

Mention may in particular be made of the compounds of formula:
R⁹-Z—(R¹⁴-Z)ₘ—R¹⁰
in which:
—R⁹ and R¹⁰ are, independently of one another, a hydrogen atom; a linear or branched alkyl radical having 1-20, preferably 1-10, carbon atoms; an aryl radical; a heterocyclyl radical; or an alkyl radical having 1-6 carbon atoms which is substituted with an alkoxy radical having 1-6 carbon atoms or a dialkylamino radical having 1-4 carbon atoms or a —C(═Y)R⁵ or —C(═Y)NR⁶R⁷ and/or YC(═Y)R⁸ radical (see the definitions R⁵ to R⁸ and Y above);
it being given that R⁹ and R¹⁰ can be joined so as to form a saturated or unsaturated
R¹⁴ represents, independently of one another, a divalent group chosen from alkanediyls having 24 carbon atoms; alkenylenes having 24 carbon atoms; cycloalkanediyls having 3-8 carbon atoms; cycloalkenediyls having 3-8 carbon atoms; arenediyls and heterocyclylenes;
Z represents O, S, NR¹⁵ or PR¹⁵, with R¹⁵ representing H; a linear or branched alkyl radical having 1-20 carbon atoms; an aryl radical; a heterocyclyl radical; or an alkyl radical having 1-6 carbon atoms which is substituted with an alkoxy radical having 1-6 carbon atoms or a dialkylamino radical having 14 carbon atoms or a —C(═Y)R⁵ or —C(═Y)NR⁶R⁷ and/or YC(═Y)R⁸ radical (see the definitions of R⁵ to R⁸ and Y above);
m is between 0 and 6.

Mention may also be made of the compounds of formula: R²⁰R²¹C[C(═Y)R⁵] in which:
R²⁰ and R²¹ are, independently of one another, a hydrogen atom; a halogen atom; a linear or branched alkyl radical having 1-20, preferably 1-10, carbon atoms; an aryl radical; or a heterocyclyl radical; it being given that R²⁰ and R²¹ can be joined so as to form a saturated or unsaturated ring; it being given that, in addition, each radical can be substituted with an alkyl radical having 1-6 carbon atoms, an alkoxy radical having 1-6 carbon atoms or an aryl radical;
R⁵ and Y being defined above.

Mention may further be made, as ligands, of carbon monoxide; optionally substituted porphyrins and porphycenes; optionally substituted ethylenediamine and propylenediamine; polyamines with tertiary amines, such as pentamethyldiethylenetriamine; aminoalcohols, such as aminoethanol and aminopropanol, which are optionally substituted; glycols, such as ethylene glycol or propylene glycol, which are optionally substituted; arenes, such as benzene, which are optionally substituted; optionally substituted cyclopentadiene; optionally substituted pyridines and bipyridines; acetonitrile; 1,10-phenanthroline; cryptands and crown ethers; or sparteine.

The preferred ligands are chosen in particular from pyridines and bipyridines which are optionally substituted by C₂-C₁₅ alkyl radicals, in particular C₆-C₁₂ radicals and especially the nonyl radical; or polyamines with tertiary amines, such as pentamethyldiethylenetriamine.

The polymerization of the monomers, in the presence of the initiator, of the compound comprising a transition metal and of the ligand which acts as activator, results in the production of a polymer having a star structure, which can be represented by the formula (I) given above, in which the monomers have polymerized to give “n” alike or different polymer chains all connected to a polyfunctional centre A which derives from the initiator.

It has been found that, in order to achieve the goal pursued by the present invention, that is to say to obtain a composition which does not exhibit the disadvantages of the prior art and which in particular allows good fixing to be obtained while retaining easy disentangling of the hair, it is preferable to choose a polymer corresponding to the following criteria:
it preferably comprises one or more monomers Mi, the corresponding homopolymer of which exhibits a Tg of greater than or equal to approximately 10° C., preferably of greater than or equal to 15° C. and even better still of greater than or equal to 20° C.;
this or these monomers Mi being present in the final polymer in an amount of between 55 and 95% by weight, preferably in an amount of 60 to 93% by weight and even better still in an amount of 65-90% by weight with respect to the total weight of monomers; and
it preferably furthermore comprises one or more monomers Mj, the corresponding homopolymer of which exhibits a Tg of less than or equal to approximately 10° C., preferably of less than or equal to 5° C. and even better still of less than or equal to 0° C.;
this or these monomers Mj being present in the final polymer in an amount of between 5 and 45% by weight, preferably in an amount of 7 to 40% by weight and even better still in an amount of 10-35% by weight with respect to the total weight of monomers.

The Tg (glass transition temperature) is measured by DSC (Differential Scanning Calorimetry) according to ASTM Standard D3418-97.

The polymers as defined in the present invention are preferably be film-forming or can be rendered film-forming by addition of an additional agent which is able to form a film. The term “film-forming” is understood to mean that the polymer, after application to a substrate and evaporation of the solvent (aqueous or organic), results in a transparent and uncracked film.

Such an additional agent which is able to form a film can be chosen from any compound known to a person skilled in the art as being capable of fulfilling the desired role and can be chosen in particular from plasticizing agents and/or from coalescence agents. Mention may in particular be made, alone or as a mixture, of:
glycols and their derivatives, such as diethylene glycol ethyl ether, diethylene glycol methyl ether, diethylene glycol butyl ether, diethylene glycol hexyl ether, ethylene glycol ethyl ether, ethylene glycol butyl ether or ethylene glycol hexyl ether-,
glycerol esters, such as glycerol diacetate (diacetin) and glycerol triacetate (triacetin);
propylene glycol derivatives, in particular propylene glycol phenyl ether, propylene glycol diacetate, propylene glycol methyl ether, propylene glycol ethyl ether, propylene glycol butyl ether, dipropylene glycol methyl ether, dipropylene glycol butyl ether, dipropylene glycol ethyl ether, tripropylene glycol butyl ether or tripropylene glycol methyl ether;
acid esters, in particular carboxylic acid esters, such as citrates, phthalates, adipates, carbonates, tartrates, phosphates or sebacates,
oxyethylenated derivatives, such as oxyethylenated oils, in particular vegetable oils, such as castor oil; or oxyethylenated silicone oils.

The amount of additional agent which is able to form a film can be chosen by a person skilled in the art on the basis of his overall knowledge so as to form a film having the desired mechanical properties while retaining, in the composition, cosmetically acceptable properties.

The polymers as defined above can be present in the medium in a form dissolved or dispersed in an aqueous, organic or aqueous/organic phase, in particular an alcoholic or aqueous/alcoholic phase.

The polymers can be present in the composition according to the invention in an amount which can be easily determined by a person skilled in the art according to the application envisaged and which can be between 1-95% by weight, on a dry basis, with respect to the total weight of the composition, preferably between 1-50% by weight and preferably between 1-20% by weight.

The compositions, in particular cosmetic compositions, according to the invention therefore additionally comprise a cosmetically acceptable medium which can be chosen by a person skilled in the art according to the application envisaged.

This medium can comprise an aqueous phase and/or a fatty phase. It is preferably composed of water or one or more cosmetically acceptable solvents, such as alcohols, or water/solvent mixtures.

The aqueous phase can comprise water and/or a thermal water and/or a spring water and/or a mineral water and/or a floral water.

Mention may be made, among acceptable organic solvents, of:
C₁-C₄ alcohols, such as ethanol, ispropanol or n-propanol;
ethers, such as dimethoxyethane;
ketones, such as acetone or methyl ethyl ketone;
lower C₁-C₃ carboxylic acid esters, such as methyl acetate or ethyl acetate.

The fatty phase can comprise conventional volatile or non-volatile oils, gums and/or waxes of animal, vegetable, mineral or synthetic origin, alone or as mixtures, in particular:
linear, branched or cyclic, volatile or non-volatile, silicone oils which are optionally organomodified; phenylated silicones; or silicone resins and gums which are liquid at room temperature;
mineral oils, such as liquid paraffin and liquid petrolatum;
oils of animal origin, such as perhydrosqualene or lanolin;
oils of vegetable origin, such as liquid triglycerides, for example sunflower, maize, soybean, jojoba, gourd, grape seed, sesame, hazlenut, apricot, macadamia, avocado, sweet almond or castor oils, triglycerides of caprylic/capric acids, olive oil, groundnut oil, rapeseed oil or coconut oil;
synthetic oils, such as purcellin oil, isoparaffins, fatty alcohols or esters of fatty acids;
fluorinated and perfluorinated oils or fluorinated silicone oils;
waxes chosen from known animal, fossil, vegetable, mineral or synthetic waxes, such as paraffin waxes, polyethylene waxes, carnauba or candelilla waxes, beeswaxes, lanolin wax, chinese insect waxes, rice wax, ouricury wax, esparto wax, cork fibre wax, sugarcane wax, japan wax, sumach wax, montan wax, microcrystalline waxes, ozokerite, the waxes obtained by the Fischer-Tropsch synthesis, silicone waxes or their mixtures.

Furthermore, the composition according to the invention can comprise adjuvants commonly used in cosmetic, in particular hair, compositions, such as cosmetic active principles, softeners, antioxidants, opacifiers, emollients, antifoaming agents, moisturizers, vitamins, fragrances, preservatives, sequestering agents, UV screening agents, ceramides, antidandruff agents, complexing agents, agents for combating hair loss, antifungal or antiseptic agents, dyes, fillers and pigments, thickeners, fixing or non-fixing polymers or conditioning polymers, propellants, basifying or acidifying agents, proteins, hydrophilic polymers, film-forming polymers, in particular in aqueous dispersion, or surfactants, in particular anionic or nonionic surfactants, which are optionally silicone surfactants.

Of course, a person skilled in the art will take care to choose this or these optional adjuvants and/or their amounts so that the advantageous properties of the composition according to the invention are not, or not substantially, detrimentally affected by the envisaged addition.

These compositions can be packaged in various forms, in particular in pump-action sprays or in aerosol containers, in order to ensure application of the composition in vaporized form or in foam form. Such packaging forms are indicated, for example, when it is desired to obtain a spray, a lacquer or a foam for fixing or treating the hair. The compositions in accordance with the invention can also be provided in the form of creams, of gels, of emulsions, of lotions or of waxes.

When the composition according to the invention is packaged in aerosol form for the purpose of obtaining a lacquer or a foam, it comprises at least one propellant which can be chosen from volatile hydrocarbons, such as n-butane, propane, isobutane, pentane, a chlorinated and/or fluorinated hydrocarbon and their mixtures. Use may also be made, as propellant, of carbon dioxide gas, nitrous oxide, dimethyl ether (DME), nitrogen or compressed air. Use may also be made of mixtures of propellants. Dimethyl ether is preferably used.

The propellant is advantageously present at a concentration of between 5 and 90% by weight with respect to the total weight of the composition in the aerosol device and more particularly at a concentration of between 10 and 60%.

The compositions in accordance with the invention can be applied to dry or wet hair.

The compositions according to the invention therefore find a very particular application as composition for treating or fixing the hair.

The invention is illustrated in more detail in the following examples.

### EXAMPLE 1

### Preparation of the Initiator

The initiator prepared was 5,11,17,23,29,35,41,47-octa(2-propionylbromide)-49,50,51,52,53,54,55,56-octa(tert-butyl)calix(8)arene (M=2378 g).

The reactants used were as follows:

| | |
|---|---|
| 4-(tert-butyl)calix(8)arene (M = 1298 g), comprising 8 | 15 g |
| phenol units (Aldrich) | |
| 2-bromopropionyl bromide of formula CH₃—CHBr—COBr | 59.9 g |
| triethylamine | 28 g |
| tetrahydrofuran (THF) | 120 g |

The 4-(t-butyl)calix(8)arene and the solvent THF were added to a round-bottomed flask equipped with a stirrer and a thermometer; the mixture was left stirring for 10 minutes at room temperature.

The triethylamine was subsequently added, which takes approximately 15 minutes.

The 2-bromopropionyl bromide, dissolved beforehand in THF, was then added at a temperature of approximately 5° C., which took approximately 1 h 30.

The mixture was left stirring for at least 12 hours at 5° C. and then the temperature was allowed to gradually rise to room temperature.

The solution obtained was concentrated by evaporating the THF. A product was precipitated from a water/ice mixture, extraction was then carried out with ethyl ether and the extract was dried over magnesium sulphate.

The solution obtained was concentrated and a compound was precipitated from a methanol/ice (90/10) mixture in a compound/precipitant ratio of 1/5.

23 g of compound were obtained, i.e., a yield of 85%, which compound existed in the form of a powder.

Characterization was carried out by NMR/GC or HPLC. The compound obtained exhibited values in accordance with those expected.

### EXAMPLE 2

### Preparation of an 8-Branched Star Polymer, Each Branch of Which was a Block Copolymer

1) First Stage: Preparation of a Star Polymer with 8 poly(tert-butyl acrylate) Branches

The reactants used were as follows:

| | |
|---|---|
| monomer 1: tert-butyl acrylate (Tg = 50° C.) | 115 g |
| monomer 2: butyl acrylate (Tg = −50° C.) | 5 g |
| initiator (prepared according to Example 1) | 1.19 g |
| (corresponding to 4 × 10⁻³ mol of RBr) | |
| CuBr (corresponding to 4 × 10⁻³ mol) | 0.57 g |
| Bipyridine (corresponding to 8 × 10⁻³ mol) | 1.25 g |

The monomers were distilled beforehand.

The reactants, except the monomers, were mixed in a sealed and flame-treated reactor comprising a nitrogen inlet and then the monomer 1 was added.

The reactor was heated under nitrogen to approximately 120° C. and reaction was then allowed to take place at 120° C. for 4 hours, the nitrogen inlet being disconnected.

2) Second Stage: Formation of the Second Block at the End of Each Branch

The monomer 2 was then added and reaction was again allowed to take place at 120° C. for 4 hours.

After reaction, the reaction mixture was allowed to cool; a viscous green solution was obtained, which solution was dissolved in dichloromethane. The polymer solution was passed through neutral alumina and the clear solution obtained was precipitated from a methanol/water (80/20) mixture in a polymer/precipitant ratio of 1/5.

115 g of polymer were obtained, i.e., a yield of 96%, which polymer existed in the form of a viscous product.

This polymer was a star polymer with 8 poly(isobutyl acrylate) branches, each branch of which was a block copolymer: calix(poly(tert-butyl acrylate)-block-poly(butyl acrylate)).

Characterization was carried out by GC:THF linear polystyrene equivalent, light scattering detection: 350,000 g/mol (theoretical mass: approximately 240,000); polydispersity index: 1.6.

The polymer obtained exhibited values in accordance with those expected.

The polymer was soluble in ethanol.

### Example 3

### Styling Composition

A preconditioning styling composition was prepared which comprises:

| | | |
|---|---|---|
| polymer of Example 1 | 7 | g |
| ethanol | q.s. for 100 | g |

An aerosol lacquer was subsequently prepared which comprises:

| | |
|---|---|
| above composition | 70 g |
| DME | 30 g |

After application of the lacquer to the hair, a good styling power was obtained, as well as a rapid drying time and good cosmetic properties, in particular with regard to disentangling and touch.

1-40. (Canceled)

41. A hair composition, comprising, in an acceptable medium, at least one polymer having a star structure chosen from structures of formula (I):
A—[(M1)ₚ₁−(M2)ₚ₂ . . . (Mi)ₚⱼ]ₙ (I)
in which:
A is chosen from polyfunctional centers having a functionality n; [(M1)ₚ₁−(M2)ₚ₂ . . . (Mi)ₚⱼ] represents a branch comprising at least one polymerized monomeric unit Mi having a polymerization index pj;
n is an integer greater than or equal to 2;
pj is greater than or equal to 2;
there are at least two branches, which may be identical or different; and
said at least two branches are grafted covalently to A;
wherein said at least one polymerized monomeric unit Mi comprised by at least one of said at least two branches is chosen from polymerized monomeric units Mk, which may be identical or different, wherein a homopolymer formed by the corresponding polymerized monomeric units Mk has a Tg of greater than or equal to 10° C.; and
wherein said at least one polymerized monomeric unit Mi contained by at least one of said at least two branches is chosen from polymerized monomeric units Mj, which may be identical or different, wherein a homopolymer formed by the corresponding polymerized monomeric units Mj has a Tg of less than or equal to 10° C.

42. A composition according to claim 41, wherein said at least one polymerized monomeric unit Mi chosen from polymerized monomeric units Mk is present in an amount ranging from 55 to 95 percent by weight relative to the total weight of the polymerized monomeric units Mi.

43. A composition according to claim 41, wherein said at least one polymerized monomeric unit Mi chosen from polymerized monomeric units Mj is present in an amount ranging from 5 to 45 percent by weight relative to the total weight of the polymerized monomeric units Mi.

44. A composition according to claim 44, wherein said at least one agent which is able to form a film.

45. A composition according to claim 44, wherein said at least one agent is chosen from plasticizing agents and coalescence agents.

46. A composition according to claim 41, wherein said at least one polymer is present in an amount ranging from 1 to 95 percent by weight, on a dry basis, with respect to the total weight of said composition.

47. A composition according to claim 46, wherein the range is from 1 to 50 percent by weight.

48. A composition according to claim 46, wherein the range is from 1 to 20 percent by weight.

49. A composition according to claim 41, wherein said at least one polymer is present in said acceptable medium containing at least one phase chosen from aqueous phases, organic phases, and aqueous/organic phases.

50. A composition according to claim 49, wherein said at least one phase is chosen from alcoholic and aqueous/alcoholic phases.

51. A composition according to claim 49, wherein said at least one polymer is dissolved or dispersed in said at least one phase.

52. A composition according to claim 41, wherein said composition has a form chosen from sprays, lacquers, foams, creams, gels, emulsions, lotions, and waxes.

53. (Canceled)

54. A composition according to claim 41, wherein said form is a composition for treating and/or fixing the hair.

55. A process for retaining or shaping the hair, comprising applying to said hair a composition, comprising, in an acceptable medium, at least one polymer having a star structure chosen from structures of formula (i):
A−[(M 1)ₚ₁−(M2)ₚ₂ . . . (Mi)ₚⱼ]ₙ (I)
in which:
A is chosen from polyfunctional centers having a functionality n; [(M1)ₚ₁−(M2)ₚ₂ . . . (Mi)ₚⱼ] represents a branch comprising at least one polymerized monomeric unit Mi having a polymerization index pj;
n is an integer greater than or equal to 2;
pj is greater than or equal to 2;
there are at least two branches, which may be identical or different; and
said at least two branches are grafted covalently to A;
wherein said at least one polymerized monomeric unit Mi comprised by at least one of said at least two branches is chosen from polymerized monomeric units Mk, which may be identical or different, wherein a homopolymer formed by the corresponding polymerized monomeric units Mk has a Tg of greater than or equal to 10° C.; and
wherein said at least one polymerized monomeric unit Mi contained by at least one of said at least two branches is chosen from polymerized monomeric units Mj, which may be identical or different, wherein a homopolymer formed by the corresponding polymerized monomeric units Mj has a Tg of less than or equal to 10° C.

56. A process according to claim 55, wherein said at least one polymerized monomeric unit Mi chosen from polymerized monomeric units Mk is present in an amount ranging from 55 to 95 percent by weight relative to the total weight of the polymerized monomeric units Mi.

57. A process according to claim 55, wherein said at least one polymerized monomeric unit Mi chosen from polymerized monomeric units Mj is present in an amount ranging from 5 to 45 percent by weight relative to the total weight of the polymerized monomeric units Mi.

58. A process according to claim 55, wherein said hair is human hair.

59. A process for preparing a styling product, comprising introducing, in an acceptable medium, at least one polymer in an amount effective for retaining and/or shaping hair, wherein said at least one polymer having a star structure chosen from structures of formula (1):
A−[(M 1)ₚ₁−(M2)ₚ₂ . . . (Mi)ₚⱼ]ₙ(I)
in which:
A is chosen from polyfunctional centers having a functionality n; [(M1)ₚ₁−(M2)ₚ₂ . . . (Mi)ₚⱼ] represents a branch comprising at least one polymerized monomeric unit Mi having a polymerization index pj;
n is an integer greater than or equal to 2;
pj is greater than or equal to 2;
there are at least two branches, which may be identical or different; and
said at least two branches are grafted covalently to A;
wherein said at least one polymerized monomeric unit Mi comprised by at least one of said at least two branches is chosen from polymerized monomeric units Mk, which may be identical or different, wherein a homopolymer formed by the corresponding polymerized monomeric units Mk has a Tg of greater than or equal to 10° C.; and
wherein said at least one polymerized monomeric unit Mi contained by at least one of said at least two branches is chosen from polymerized monomeric units Mj, which may be identical or different, wherein a homopolymer formed by the corresponding polymerized monomeric units Mj has a Tg of less than or equal to 10° C.

60. A process according to claim 59, wherein said at least one polymerized monomeric unit Mi chosen from polymerized monomeric units Mk is present in an amount ranging from 55 to 95 percent by weight relative to the total weight of the polymerized monomeric units Mi.

61. A process according to claim 59, wherein said at least one polymerized monomeric unit Mi chosen from polymerized monomeric units Mj is present in an amount ranging from 5 to 45 percent by weight relative to the total weight of the polymerized monomeric units Mi.
